(19) United States
(12) Patent Application Publication
(10) Pub. No.: US 2022/0167693 A1
(43) Pub. Date: Jun. 2, 2022

(54) Face Mask with Applied Emitters

(71) Applicant: Jeremy Madvin, Westlake Village, CA (US)

(72) Inventor: Jeremy Madvin, Westlake Village, CA (US)

(21) Appl. No.: 17/108,135

(22) Filed: Dec. 1, 2020

(51) Int. Cl. A41D 13/11; A61L 9/22

(52) U.S. Cl. CPC A41D 13/1161; A61L 2209/134; A61L 9/22

(57) ABSTRACT

A wearable breathing mask configured inform of an anion mask with a material such as, but not limited to cotton, nylon, polyethene, polypropylene or some other material used to produce textiles or another suitable breathable substrate. This material acts as a substrate displaying distanced anion emitters completely covering the nostril and mouth area to ensure the constant flow of anions but avoid the avoid repulsion or unflattering appearance. The frame comprises a body with construction or openings to isolate the air circulating within the mask, an adjustable head band to fit on the user's face. The anion emitters may be made by including but not limited to materials such as tourmaline, germanium, or other natural or unnatural emitting materials. The advantages of using the various models of the masks includes helpful in solving numerous problems including but not limited to limitation of Antibacterial effect of anions, antiviral effect of anions, Anti-allergic effects of anions and Anti-asthma effects of anions. The assembly is simple, lightweight, adjustable and comfortable to use similar to normal masks. User can easily get used to inhale thru nose and exhale through mouth or vice versa.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The present invention relates to a wearable filtering face masks. The invention in particular focuses on face mask with multiple benefits to the users.

### Description of the Related Art

Today, a variety of clinical diseases occur due to pathogens like bacteria, fungi and viruses that are transmitted through air. Air transmission can be brought about by coughing, sneezing, talking or aerosol generation in any other way. The World Health Organization (WHO) has attributed the transmission of airborne diseases to the spread of droplet nuclei that persist in the air for a long time and can cover a long distance to cause diseases. This type of transmission can be further categorized into obligate or preferential. The former being confined to only droplet transmission and the later possessing multiple ways of spreading.

Face masks are a symbol of the pandemic era—a visual metaphor for the tiny, unseen viral foe that could be lurking around any corner. Furthermore, air pollution has become an increasing threat to humans due to industrialization and de-forestation, resulting in increasing cases of human respiratory illnesses, pollution related complications of cardiovascular conditions and conditions related to the organs, resulting in an increase in health care costs. Wearing a mask in daily life is becoming a norm in many places around the world.

Some opt for a scarf wrapped around their face, others make do with a t-shirt yanked up over their mouth. The more creative might hook colorful homemade varieties around their ears, while a lucky few wear distinctive surgical masks or, rarer still, N95 respirators.

While a few months ago anyone wearing a mask in public would have drawn stares in many countries not used to this behavior, they are now a reminder of the strange times we live in. And as governments around the world start to ease their lockdowns to allow their citizens out to mingle in the wider world again, growing numbers of people are opting to wear face masks in public.

There are multiple types of enhancements that can be seen in prior art regarding face masks. For instance a Multipurpose therapeutic face mask bearing US patent 2,013,0276784A1 is issued to Stephen Donald Flynn, SR. The patent is on a therapeutic face mask comprises a face-engaging portion and a single connector having a mask-engaging end and a single treatment-receiving end which has a single attachment mounting for detachable sealingly receiving a treatment attachment, such as an oxygen reservoir bag, or a nebulizer. A one-way inhalation valve in the connector permits fluid flow from the treatment-receiving end to the mask-engaging end during inhalation and inhibits fluid flow in the other direction. The mask also includes a valve-governed exhalation port and an anti-asphyxia valve assembly configured to permit fluid flow from ambient to the face-engaging portion during inhalation only when inspiratory effort during inhalation exceeds fluid flow to the treatment-receiving end of the connector. Also provided is an oxygen reservoir bag having a neck shaped for removable coupling to a mating connector of a therapeutic face mask. An oxygen reservoir bag may have a metered-dose inhaler port defined in its neck.

Another patent on Adjustable facial conforming face mask bearing US patent 2,017,0095681A1 is issued to BALBOA MANUFACTURING COMPANY LLC. The patent is on an adjustable face mask having a body conforming substantially to the facial structure of a wearer. The face mask is provided with a neck protector and removable filter and with a strip of fabric designed to push air down to the sides of the wearer's face in order to eliminate fogging of the wearer's goggles. The face mask can optionally provide neck protection with a descending neck member.

Another patent on Full-face mask and mask cushion therefor bearing US patent 2,014,0360506A1 is issued to Resmed Pty Ltd. The patent is on a full-face cushion comprises a substantially triangularly shaped frame from which extends a membrane. The frame has a scalloped edge by which the cushion is affixed to a mask body to form a full-face mask. The membrane has an aperture into which the wearer's nose is received. The member is spaced away from the rim of the frame, and its outer surface is of substantially the same shape as the rim. Respective notches receive the bridge of the wearer's nose. The wearer's nose and lips are received through the aperture into the chamber within the mask body. The seal forming portion thus contacts both the surface of the wearer's nose and a portion of the wearer's face in the region between the bottom lip and the chin, and around the sides and over the bridge of the nose. The shape of the seal forming portion is particularly suited to effectively seal the difficult region of the facial contour that is the crease between the sides of the nose and the face.

Another Face mask bearing US patent 2,016,0113336A1 is issued to Uni Charm Corp. The patent is on a mask has a mask body and a pair of ear straps. The mask body includes an outer layer sheet and an intermediate layer sheet. The outer layer sheet is formed of hydrophobic fibers. The intermediate layer sheet is laid on the outer layer sheet so as to be located on a wearer's side of the outer layer sheet when the mask is worn. The intermediate layer sheet includes a first fiber layer which is formed of polyolefin fibers containing an inorganic antimicrobial agent and a second fiber layer which is formed of polyolefin fibers and has a larger fiber diameter than the first fiber layer. The fiber diameter of the first fiber layer is within a range of 0.5 to 2.8 μm and the ratio of a particle diameter of the inorganic antimicrobial agent with respect to the fiber diameter is within the range of 0.1 to 6.0.

A Cold Weather Exposure Mask bearing US patent 2,014,0283280A1 is issued to TALUS OUTDOOR TECHNOLOGIES LLC. The patent is on a Cold or Inclement Weather Exposure Mask is designed to fit snugly around the wearer's face, head, and neck. The mask serves vital needs of warmth, humidity, moisture management and air flow. A raised, pliable chamber covering the mouth and nose of the wearer provides a breathing space to allow normal breathing while keeping the face warm and dry. The chamber is affixed to a fabric panel made of a water and wind resistant, non-irritating, comfortable, and stretchable material. A fastening means on the panel holds the mask in place around the wearer. The chamber includes multiple apertures which allow fresh, ambient air to pass to the wearer after it is mixed with warm, humid, exhaled air. The apertures also exhaust breath, air and condensation. Colors and pattern selections allow adaptation to all labor and recreation uses.

Another patent on Respiratory mask assembly bearing U.S. Pat. No. 8,479,736B2 is issued to Resmed Pty Ltd. The patent is on a respiratory mask assembly for delivering breathable gas to a patient includes a frame having a main body and a side frame member provided on each lateral side of the main body. Each side frame member includes an integrally formed first connector portion. A headgear assembly is removably attachable to the frame. The headgear assembly has a second connector portion adapted to be removably coupled with the first connector portion provided on the frame. The second connector portion is manually movable to a releasing position to detach the headgear assembly from the frame. The headgear assembly is rotationally adjustable with respect to the frame.

A Mask system bearing U.S. Pat. No. 8,528,561B2 is issued to Resmed Pty Ltd. The patent is on a mask includes a shroud; a cushion module that engages the shroud, the cushion module including a frame and a full-face cushion adapted to engage the patient's face generally along nasal bridge, cheek, and chin regions of the patient's face, the frame at least partly defining a breathing chamber, an elbow connected to the frame in a releasable and rotatable manner, the elbow being adapted to be connected to an air delivery tube to deliver breathable gas to the patient; and headgear removably attachable to the shroud, wherein: the fame includes an opening, the elbow including a distal end releasably inserted into the opening of the frame with a snap-fit, thereby connecting the elbow to the frame, whereby the connection between the elbow and the frame also maintains the shroud in engagement with the frame.

There are multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them unsuitable for many users. Moreover, the ease of use of these assemblies is also questionable.

It has to be noted that the current invention proposes an assembly with an advancement where the design adds an element of anion emission displaying intentionally distanced anion emitters to a material such as, but not limited to cotton, nylon, polyethene, polypropylene or some other material used to produce textiles or another suitable breathable substrate.

The substrate must cover the nostril and mouth area to ensure the constant flow of anions but avoid the avoid repulsion or unflattering appearance. Each anion emitter is capable of constant and consistent emission of anions without interruption. Each emitter is durable enough to withstand dozens, if not hundreds of wash cycles. Each emitter operates without batteries of other external power sources. Each cubic centimeter of spotted material offers thousands of active anions at all times.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a face mask with multiple enhanced functionalities.

Another object of the present invention is to provide a face mask, comprising a frame having an exterior surface, a breathable interior surface, wherein the frame is adapted to overlay a user's mouth and nose; an outer breathable layer overlaying the face mask and having a pair of straps with inter-engaging ends for affixing the frame about the user's face.

Another object of this invention is to provide a new and improved frame which is designed to be worn on a wearer's face and includes at least a mask body and a pair of ear straps for securing the mask to the users face. The frame and substrate may be designed for a single or multiple use which can be used once or several times, or reusable type which can be reused by washing with soap and water.

It is also the objective of the invention to provide Anion based technology which has the potential to reduce viral load from the air.

It is also the objective of the invention to provide assembly where Anions have the ability to reduce allergenic effects generated by several allergens

It is further the objective of the invention to provide negative ions which has added airborne particulate reducing power and can maintain biological protection. They are also considered as the vitamins of air that have advantageous effects on human health. It further assists the body to maintain homeostasis.

It is another objective of the invention to provide a support frame assembly with variety of design options so user can choose as per his/her requirements.

It is another object to provide a reliable construction which includes the mask proposed to be made out of with a material including but not limited to cotton, nylon, polyethene, polypropylene or some other material used to produce textiles or another suitable breathable substrate.

The substrate must cover the nostril and mouth area to ensure a proper filtering barrier and the constant flow of anions but avoid repulsion or unflattering appearance. This frame and substrate are ideal for both females and males, suitable for cycling, camping, running, travel, climbing as well as medical environments and daily use.

Yet another object of the present invention is to provide a structure that is relatively inexpensive to produce so that ownership is available to a substantial majority of families.

It is also the objective of the invention to provide a smart assembly which provides ease of use and convenience to everyone.

It is a further aspect of the present invention to provide a new and improved assembly which is of a durable and reliable construction and can sustain its effectiveness after multiple and consistent use.

Thus, it is the objective to provide a new and improved frame and substrate. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 is a perspective view of a facemask showing my new design;

FIG. 2 is a left side elevation view thereof;

FIG. 3 is a right-side elevation view thereof;

FIG. 4 is a front elevation view thereof;

FIG. 5 is a rear elevation view thereof;

FIG. 6 is a top plan view thereof;

FIG. 7 is a bottom plan view thereof;

FIG. 8 is a perspective view of a facemask showing another embodiment of my new design;

FIG. 9 is a left side elevation view thereof;

FIG. 10 is a right-side elevation view thereof;

FIG. 11 is a front elevation view thereof;

FIG. 12 is a rear elevation view thereof;

FIG. 13 is a top plan view thereof; and

FIG. 14 is a bottom plan view thereof.

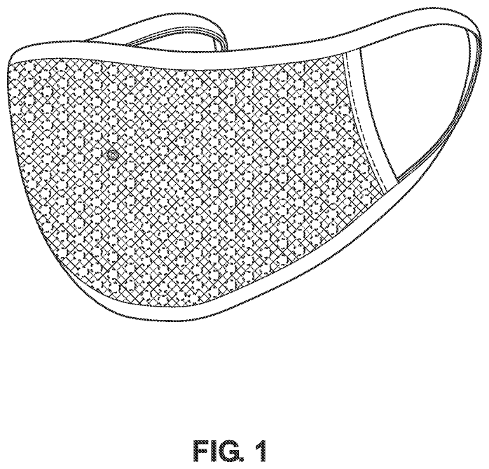

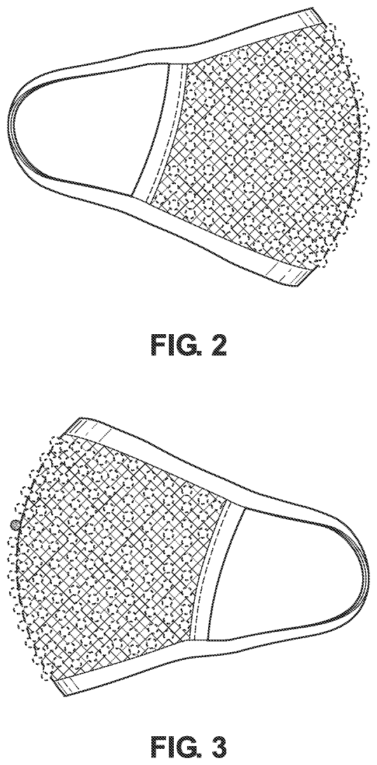

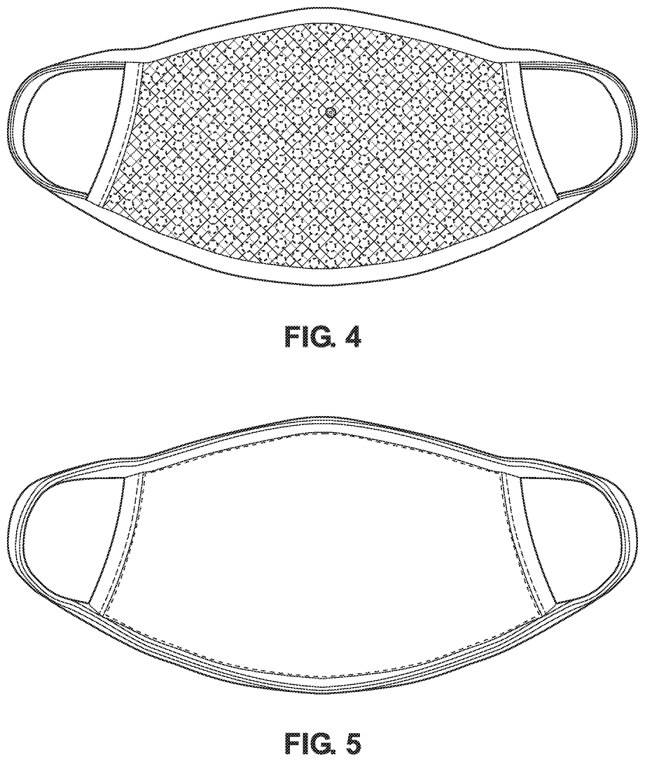

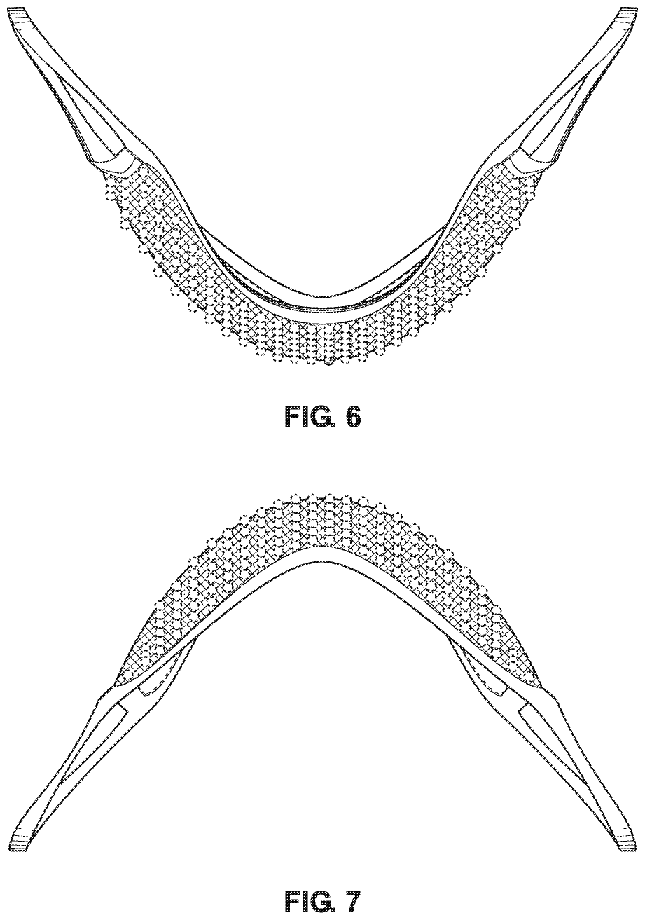

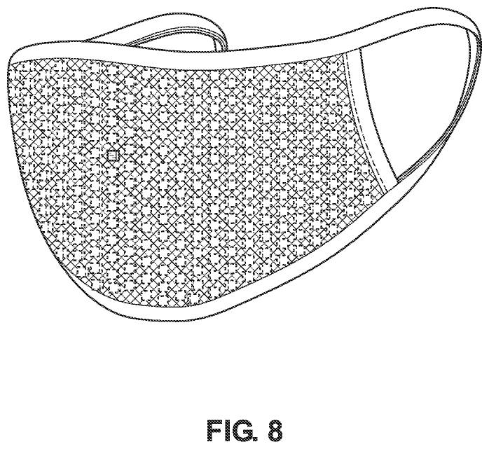

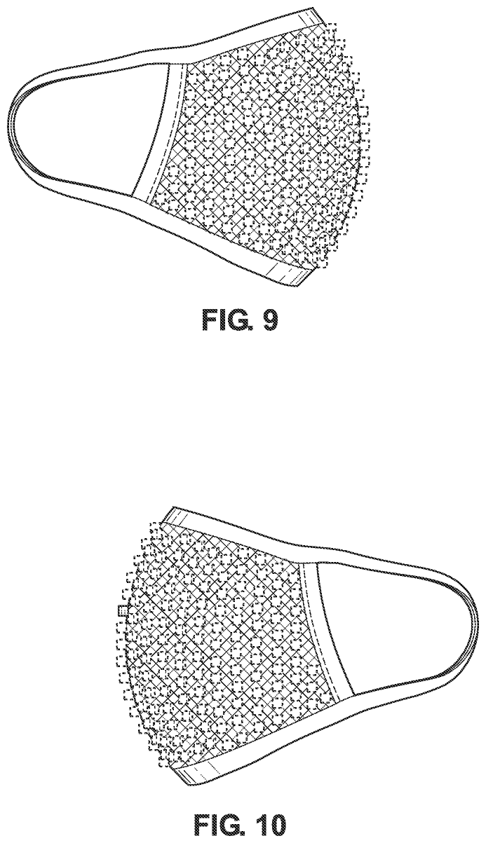

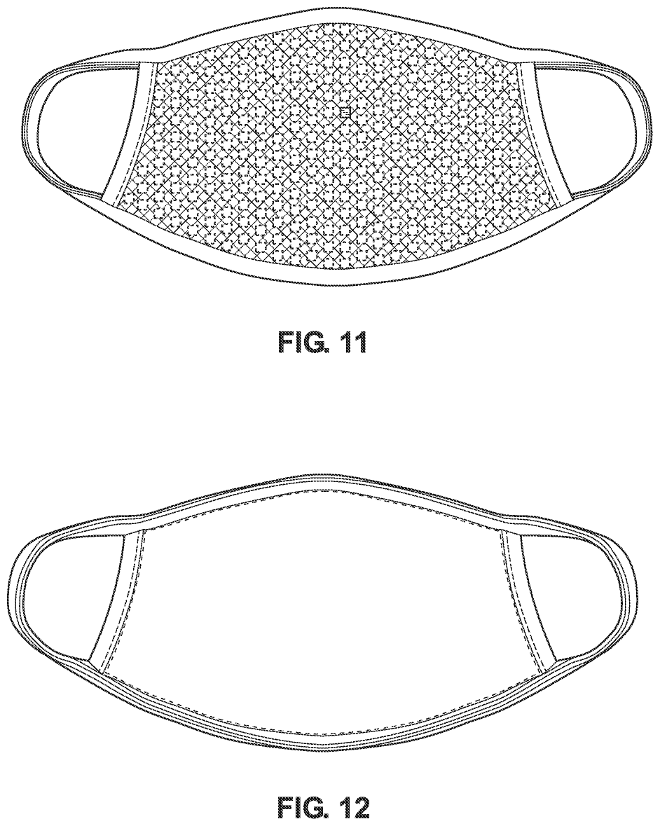

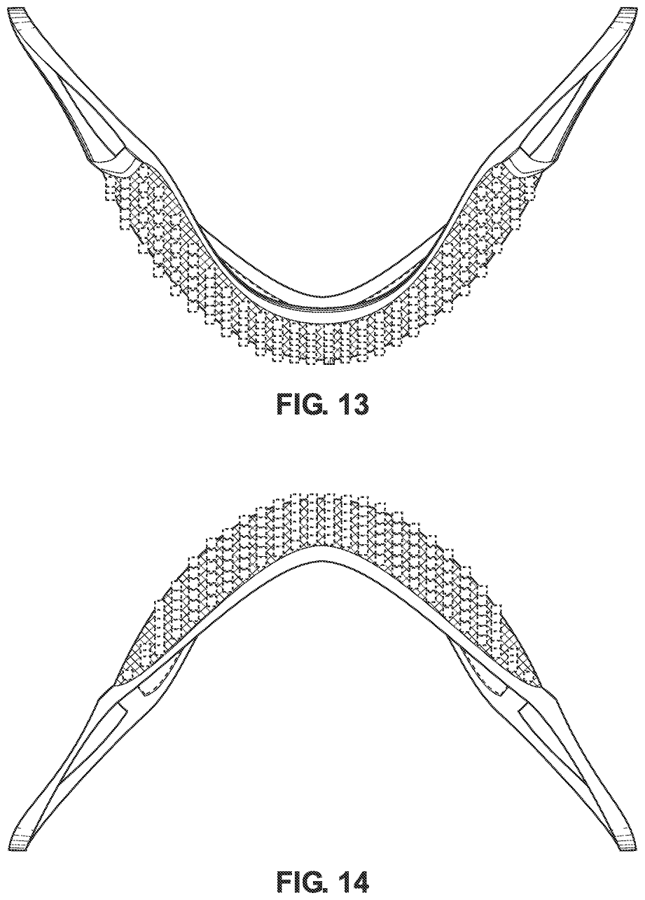

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to provide an improved face mask, comprising a frame assembly having a breathable exterior surface, a breathable interior surface, wherein the face mask is adapted to overlay a user's mouth and nose; an outer layer overlaying the face mask and having a pair of straps with either ear loops or inter-engaging ends for affixing the face mask about the user's face; and breathable material for easy inflow and outflow of air.

Air ionization is achieved in several ways; the most common are; alpha ionization and corona ionization. The former approach utilizes polonium 210 or any other radioactive element that upon collusion with the air particles, leads to a disturbance of molecules, electron transfer and subsequent generation of negative and positive ions in equal amount. The latter approach makes use of high voltage electric current and generates ions in the air. In this process a metallic needle-like apparatus is used, that detaches the electrons from itself, with the passage of current and transfers them to the oxygen or nitrogen gas in the air, creating negative ions. These ions in turn stick to the dust particles or other unwanted units in the air, increasing their weight and making them fall down, leading to clearer air.

This type of ionization can be performed by AC or DC current application. An AC current based technique generates negative and positive ions by an emitter. In the case of a DC current based approach, separate current supplies are maintained, and bipolar ions are created. An AC based technique is suitable for component cleaning during assembly while a DC based approach can ensure cleanrooms.

The exposure of a conductive material to ultraviolet rays can turn it into an anion emitter. This approach makes use of an ultraviolet lamp and was patented in 1964. The radiations cause the emission of electrons from the material that upon collusion with air leads to the production of NAIs.

The claimed product as per its preferred embodiments is an anion mask with a material such as, but not limited to cotton, nylon, polyethene, polypropylene or some other material used to produce textiles or another suitable breathable substrate. This material displays small, intentionally distanced anion emitters completely covering the nostril and mouth area to ensure the constant flow of anions. Each anion emitter is capable of constant and consistent emission of anions without interruption. Each emitter is durable enough to withstand dozens, if not hundreds of wash cycles. Each emitter operates without batteries or other external power sources. Each cubic centimeter of spotted material offers thousands of active anions at all times.

As per further embodiments of the invention, the benefits of face masks and air ionization can be helpful in solving numerous issues including but not limited to Antibacterial effect of anions where microorganisms can now be inactivated through desiccators, heaters, chemicals, ultraviolet radiation, and ionizers. The carbon fiber-based ionizers ease up the air purification by a generation of unipolar ions. This technique has been tested against various bacteria like Escherichia coli and Staphylococcus epidermidis.

The Antiviral effect of anion-based technology has the potential to reduce viral consequences. In a study, Canine calicivirus and rotavirus aerosol particles were collected from the air by means of an ionizer. Viral particles of variable sizes can be inactivated in this way.

The invention as per its further embodiments allows Anions to have been proven to have the ability to reduce allergenic effects generated by several allergens. A research study was conducted by using Japanese cedar pollen extract after being exposed to ions. As a result, the IgE from allergic patients was unable to bind the allergen. It was assumed that the allergic response could be minimized up to 80% by using this treatment.

The administration of negative ion therapy has been explored in relation to asthma as well. In children shorter spasms were reported when they were treated with anions as opposed to conventional medicines. Abnormal and rapid breathing was minimized in cases with bronchopneumonia and bronchitis. The positive outcomes were reported within eight hours of therapy.

The Negative ions have an airborne particulate reducing power and can maintain biological protection. They can be considered as the vitamins of air that have advantageous effects on human health. They assist the body to maintain homeostasis.

Moreover, the anions may participate in the healing of damaged cells and contribute towards the activation of cell vitality. Negatively charged ions can regulate the nervous system, improve respiration, and blood flow. They can also prevent, mitigate, or eliminate respiratory ailments.

The Serotonin, mediated by 5-hydroxytryptophan, is produced by nerve cells if the number of cations entering the body is more than the anions. Serotonin upon transportation to the tissues, hinders the oxygen absorption into the blood stream. The consequences of this incident may range from regular migraines, dizziness, fatigue, depression and even breathing issues. Hence there is a need to administer the negative ions through nose, mouth, or skin. They can assist the conversion of 5-hydroxytryptophan to a harmless compound that can be excreted through urine.

The putative anxiety disorder termed as serotonin irritation syndrome can be reversed by the use of anions.

Furthermore, these negatively charged ions can actively promote the endocrine function and associated metabolism. They can also improve the immune system and enhance a person's working capacity. They can act as mood stabilizers, relaxers and can aid in the treatment of hypertension, heart diseases and depression.

The invention as per its further embodiments has tools that run on the principle of negative ion generation can in addition to potential reduction of viruses and bacteria, appropriately dispel the dust particles, pollutants, smoke, pollens, fungal spores and air borne allergens from the air.

In an embodiment, of the present invention the advantages including but not limited to allowing the protection from pollution by filtering out majority of the particles through its multiple layers, very soft fabric at the back to provide skin safety, removable and adjustable body to facilitate the wearer. It is a light weight, adjustable and comfortable assembly.

The anion emitters may be made by including but not limited to materials such as tourmaline, germanium, or other natural or unnatural emitting materials.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A face mask assembly with partition wall design, comprising:
A simple support frame and substrate to be used as surgical or cloth masks to allow comfortable spacing between mask and mouth, and the support frame, comprising a frame having a breathable exterior surface, a breathable interior surface, wherein the frame is adapted to overlay a user's mouth and nose; and having a pair of straps with ear loops or inter-engaging ends for affixing the frame about the user's face and mask body with breathable material allowing the safe exit of air from the mask assembly and avoiding circulation of air within the mask and an anion mask with a material displaying small, intentionally distanced anion emitters completely covering the nostril and mouth area to ensure a proper barrier and the constant flow of anions.
